(19) United States
(12) Patent Application Publication
(10) Pub. No.: US 2009/0270708 A1
(43) Pub. Date: Oct. 29, 2009

(54) SPORTS CLOTHES

(75) Inventors: CHIEN-LUNG SHEN, Taipei Hsien (TW); Kun-Chuan Tsai, Taipei Hsien (TW); Wen-Kuei Lai, Taipei Hsien (TW)

Correspondence Address: J C PATENTS, 4 VENTURE, SUITE 250, IRVINE, CA, 92618, US

(73) Assignee: Taiwan Textile Research Institute, Taipei Hsien (TW)

(21) Appl. No.: 12/141,558

(22) Filed: Jun. 18, 2008

(30) Foreign Application Priority Data
Apr. 25, 2008 (TW) 97115357

(51) Int. Cl. A61B 5/04; A41D 3/00; A41D 1/04; A41F 19/00

(52) U.S. Cl. 600/389; 600/388; 600/386; 2/93; 2/102; 2/300

(57) ABSTRACT

A sports clothes including an inner layer structure and an outer layer structure is suitable to be worn on a human body. The inner layer structure includes a main part, an elastic structure, at least two fabric electrodes, and at least one fastener. The elastic structure is connected with the main part. The two fabric electrodes suitable for contacting with the skin of the human body are directly knitted on the elastic structure. The fastener is nailed on the elastic structure and electrically connected with the two fabric electrodes. The parts of the outer layer structure and that of the inner layer structure corresponding to at least one of the shoulder, partial neck portion, and armpit are connected. The outer layer structure covers the inner layer structure completely.

### CROSS-REFERENCE TO RELATED APPLICATION

This application claims the priority benefit of Taiwan application serial no. 97115357, filed on Apr. 25, 2008. The entirety of the above-mentioned patent application is hereby incorporated by reference herein and made a part of this specification.

### BACKGROUND OF THE INVENTION

1. Field of the Invention

The present invention generally relates to a sports clothes, in particular, to a sports clothes capable of be connected with a sensing device.

2. Description of Related Art

In recent years, the mass begins to pay more and more attention to the health and body figure, so sports has become increasingly popular, in which jogging, cycling, and other sports capable of achieving aerobic effects are especially prevailing. The aerobics is defined as a form of exercise in which a heart rate of the body is maintained between 120 and 160 (beats) per minute and the exercise is sustained for over 40 minutes. However, when the heart rate is too high, the body may enter the anaerobic exercise, and as a result, no fat is consumed.

In the conventional art, a sports clothes capable of being connected with a sensing device is provided, in which the sensing device is installed on an outer surface of the sports clothes. However, when human being does exercises, it is easy for the sports clothes to slip or shift around, so as to affect a sensing accuracy of the sensing device. The exposed sensing device connected with the sports clothes influences the entire aesthetic feeling of the sports clothes.

### SUMMARY OF THE INVENTION

Accordingly, the present invention is directed to a sports clothes, which is fixed in a better way and achieves desirable aesthetic feeling after being connected with a sensing device.

The present invention provides a sports clothes, which is suitable for being worn on a human body. The sports clothes includes an inner layer structure and an outer layer structure. The inner layer structure includes a main part, an elastic structure, at least two fabric electrodes, and at least one fastener. The elastic structure is connected with the main part. The two fabric electrodes suitable for contacting with the skin of the human body are directly knitted on the elastic structure. The fastener is nailed on the elastic structure and electrically connected with the two fabric electrodes. The parts of the outer layer structure and that of the inner layer structure corresponding to at least one of the shoulder, partial neck portion, and armpit are connected. The outer layer structure covers the inner layer structure completely.

In an embodiment of the present invention, the sports clothes further includes a sensing device, fixed on the elastic structure through the fastener, and electrically connected with the fabric electrodes through the fastener.

In an embodiment of the present invention, the parts of the outer layer structure and that of the inner layer structure corresponding to at least one of the shoulder and the partial neck portion of the human body are directly stitched together.

In an embodiment of the present invention, the part of the outer layer structure and that of the inner layer structure corresponding to the armpit of the human body are connected together by point stitching or through an elastic tape.

In an embodiment of the present invention, the elastic structure is directly machine-sewed or laminated to the main part.

In an embodiment of the present invention, the main part of the inner layer structure includes an upper part and a lower part connected with each other by the elastic structure.

In an embodiment of the present invention, the inner layer structure further includes an anti-slip structure, covering a surface of the elastic structure.

In an embodiment of the present invention, the anti-slip structure partially covers the surface of the elastic structure or completely covers the surface of the elastic structure.

In an embodiment of the present invention, the fastener is a conductive fastener, and the inner layer structure further includes an insulating structure, covering the conductive fastener.

In an embodiment of the present invention, a front side of the fastener is a conductive material, and a back side of the fastener is an insulating material.

In an embodiment of the present invention, the inner layer structure is in a style of a sleeveless coat or a vest.

In an embodiment of the present invention, the outer layer structure is in a style of a sleeve coat.

The present invention provides a sports clothes, which is suitable for being worn on a human body. The sports clothes includes a main part, an elastic reinforcement structure, an elastic structure, at least two fabric electrodes, and at least one fastener. The elastic reinforcement structure is stitched on a part of the main part corresponding to a connection part between an arm and a body of the human body. The elastic structure is connected with the main part. The two fabric electrodes suitable for contacting with the skin of the human body are directly knitted on the elastic structure. The fastener is nailed on the elastic structure and electrically connected with the at least two fabric electrodes.

In an embodiment of the present invention, the sports clothes further includes a sensing device, fixed on the elastic structure through the fastener, and electrically connected with the fabric electrodes through the fastener.

In an embodiment of the present invention, the sports clothes further includes a beautifying structure, stitched on the main part, so as to cover the sensing device.

In an embodiment of the present invention, the elastic structure is directly machine-sewed or laminated to the main part.

In an embodiment of the present invention, the main part includes an upper part and a lower part connected with each other by the elastic structure.

In an embodiment of the present invention, the sports clothes further includes an anti-slip structure, covering a surface of the elastic structure.

In an embodiment of the present invention, the anti-slip structure partially covers the surface of the elastic structure or completely covers the surface of the elastic structure.

In an embodiment of the present invention, the fastener is a conductive fastener, and the sports clothes further includes an insulating structure, covering the conductive fastener.

In an embodiment of the present invention, a front side of the fastener is a conductive material, and a back side of the fastener is an insulating material.

In an embodiment of the present invention, the main part is in a style of a sleeve coat.

To sum up, the outer layer structure of the present invention completely covers the inner layer structure and the sensing device, so the outer layer structure has an effect of beautifying the sports clothes. In addition, through the elastic reinforcement structure on the sports clothes of the present invention, the main part is not easily slipped or shifted due to being affected by the arms when the human being does exercises with the arms, thereby enhancing the sensing accuracy of the sensing device disposed on the main part.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1A is a schematic view of a sports clothes according to an embodiment of the present invention.

FIG. 1B is a schematic view of an inner layer structure of the sports clothes of FIG. 1A.

FIG. 1C is a schematic cross-sectional view of FIG. 1B, taken along a line segment I-I′.

FIG. 2A is a schematic view of a front side of a sports clothes according to another embodiment of the present invention.

FIG. 2B is a schematic view of a back side of the sports clothes of FIG. 2A.

FIG. 2C is a schematic cross-sectional view of FIG. 2A, taken along a line segment I-I′.

FIG. 3 is a schematic view of a front side of a sports clothes according to still another embodiment of the present invention.

### DESCRIPTION OF THE EMBODIMENTS

Reference will now be made in detail to the present embodiments of the invention, examples of which are illustrated in the accompanying drawings. Wherever possible, the same reference numbers are used in the drawings and the description to refer to the same or like parts.

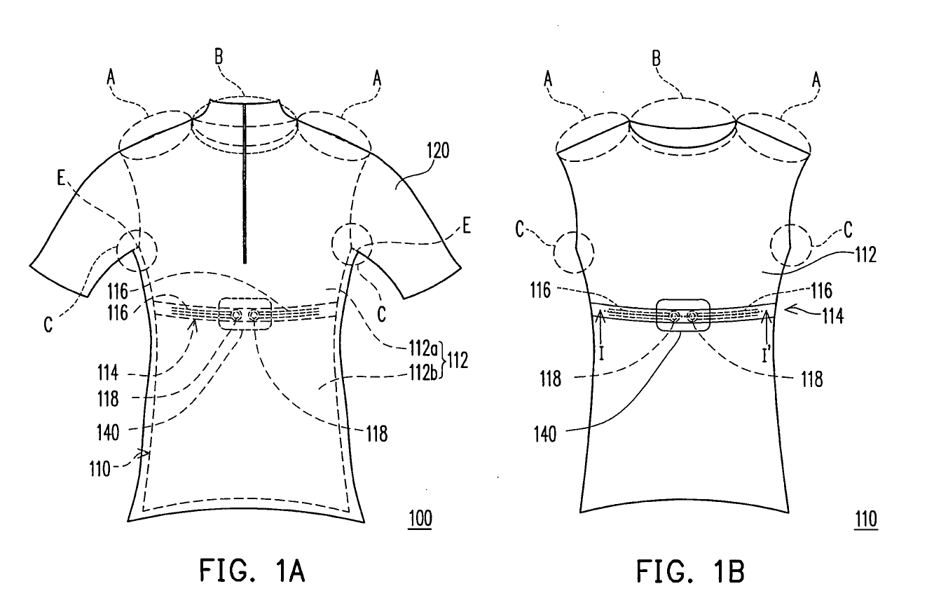

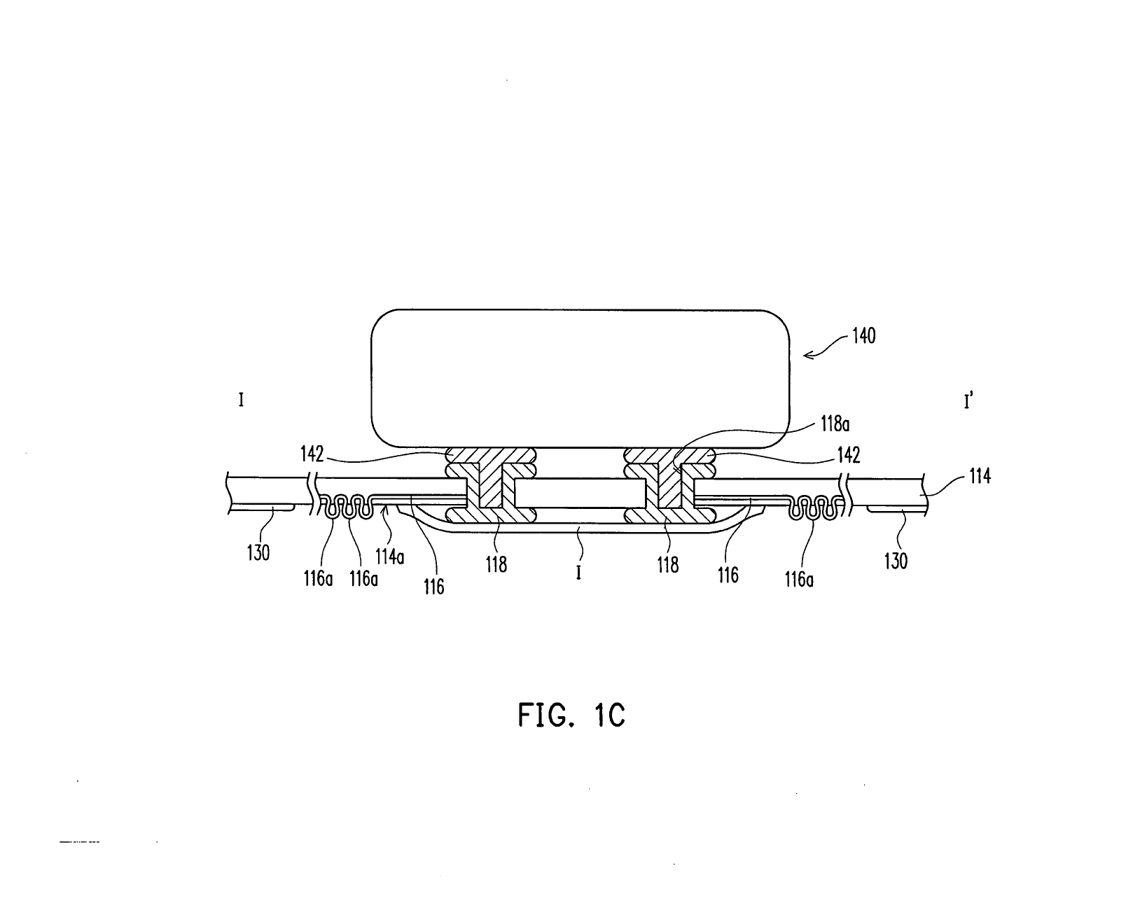

FIG. 1A is a schematic view of a sports clothes according to an embodiment of the present invention, FIG. 1B is a schematic view of an inner layer structure of the sports clothes of FIG. 1A, and FIG. 1C is a schematic cross-sectional view of FIG. 1B, taken along a line segment I-I′.

Referring to FIGS. 1A and 1B, a sports clothes 100 of this embodiment is suitable for being worn on a human body (not shown), and the sports clothes 100 may be cycling clothes or jogging clothes. The sports clothes 100 includes an inner layer structure 110 and an outer layer structure 120. The inner layer structure 110 is in a style of, for example, a sleeveless coat or a vest, and the outer layer structure 120 is in a style of, for example, a sleeve coat.

The inner layer structure 110 includes a main part 112, an elastic structure 114, two fabric electrodes 116, and two fasteners 118. It should be noted that, the number of the fabric electrodes 116 and the number of the fasteners 118 are not limited in this embodiment, for example, more than two fabric electrodes 116, and one or more fasteners 118 may be configured.

The elastic structure 114 is connected with the main part 112. In this embodiment, the main part 112 of the inner layer structure 110 includes an upper part 112a and a lower part 112b connected with each other by the elastic structure 114. In addition, in other embodiments, the elastic structure 114 may also be directly machine-sewed or laminated to the main part 112. The main part 112 is made of, for example, a high-elastic mesh fabric or another suitable high-elastic fabrics. The elastic structure 114 may be made of elastic yarns or another suitable elastic material, thereby being fixed on a particular part (for example, trunk) of the human body through utilizing the elasticity thereof.

In addition, referring to FIG. 1C, in order to make the elastic structure 114 be more stably fixed on the particular part of the human body, an anti-slip structure 130 is further configured to cover a surface 114a of the elastic structure 114 contacting with the human body, so as to prevent the elastic structure 114 from slipping or shifting while the human being does sports. In this embodiment, the anti-slip structure 130 partially covers the surface 114a of the elastic structure 114, and for example, the anti-slip structure 130 may be configured into a suitable shape, such as a strip shape and a spot shape, for being disposed on the surface 114a. In an alternative embodiment, the anti-slip structure 130 may also completely cover the surface 114a of the elastic structure 114. In another embodiment, the anti-slip structure 130 may also clad on the elastic yarns of the elastic structure 114.

The two fabric electrodes 116 suitable for contacting with the skin of the human body are directly knitted on the elastic structure 114. In this embodiment, the fabric electrodes 116 may be linear and has a plurality of annular structures 116a, in which the annular structures 116a may be protruded out of the surface 114a and contact with the human body. The fabric electrodes 116 are made of, for example, conductive yarns or another suitable conductive material.

The fasteners 118 are nailed on the elastic structure 114 and electrically connected with the two fabric electrodes 116. The fasteners 118 are conductive fasteners, and in this embodiment, an insulating structure I further covers the conductive fasteners, so as to prevent the conductive fasteners from directly contacting with the human body. Definitely, in other embodiments, a front side of the fasteners 118 may be a conductive material, and a back side thereof may be an insulating material, in which the back side is a surface contacting with the human body, and the front side is the other surface away from the human body.

In addition, referring to FIGS. 1B and 1C, in this embodiment, a sensing device 140 is further disposed on the sports clothes 100, which is fixed on the elastic structure 114 through the fasteners 118 and electrically connected with the fabric electrodes 116 through the fasteners 118. The sensing device 140 is, for example, an electrocardiogram sensing device, a respiration sensing device, a heartbeat sensing device, an electromyography sensing device, or other physiology signal sensing devices. In this embodiment, the fastener 118 has a recess 118a, and the sensing device 140 has a conductive protrusion 142 suitable for being engaged with the recess 118a. The sensing device 140 is fixed on the elastic structure 114 by engaging the conductive protrusion 142 with the fastener 118. In addition, in other embodiments, the sensing device 140 may have a recess, and the fastener 118 may have a conductive protrusion suitable for being engaged with the recess.

Referring to FIGS. 1A and 1B again, the parts A, B, and C of the outer layer structure 120 and that of the inner layer structure 110 corresponding to at least one of the shoulder, partial neck portion, and armpit of the human body are connected, and the outer layer structure 120 completely covers the inner layer structure 110. The parts A and B of the outer layer structure 120 and that of the inner layer structure 110 corresponding to at least one of the shoulder and the partial neck portion of the human body are directly stitched together. The part C of the outer layer structure 120 and that of the inner layer structure 110 corresponding to the armpit of the human body are connected together by point stitching or through an elastic tape E. When the human being does sports with the arms, the elastic tape E may absorb a part of the tension on the inner layer structure 110 applied by the outer layer structure 120, so as to prevent the outer layer structure 120 from over-pulling the inner layer structure 110.

Accordingly, the sensing device of this embodiment is disposed on the inner layer structure 110, and the outer layer structure 120 completely covers the inner layer structure 110. Therefore, when observing the sports clothes 100 from the exterior, the sensing device and the inner layer structure 110 cannot be seen at all, so the outer layer structure 120 can beautify the sports clothes 100.

Besides the double-layer structure with the inner layer and the outer layer, the present invention further provides a single-layer sports clothes, which is described below in detail.

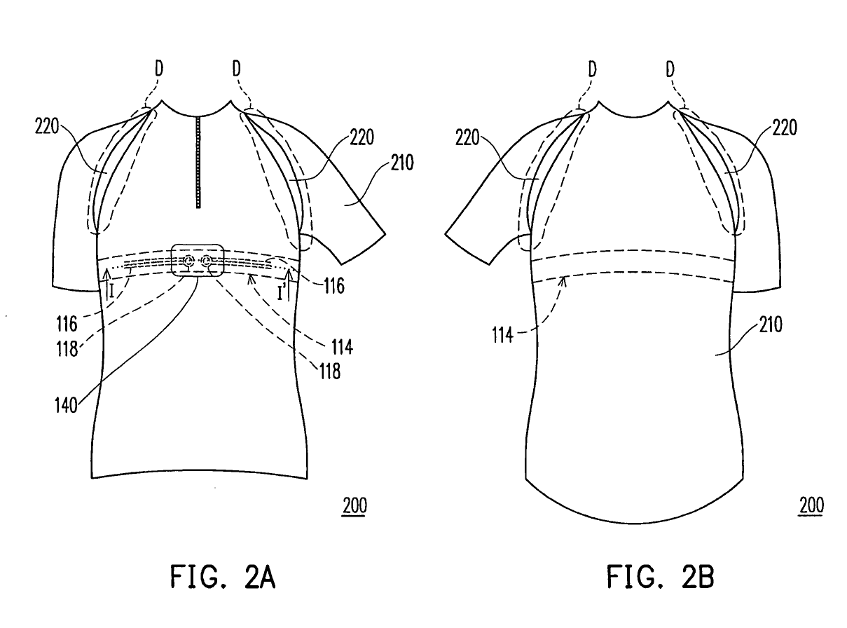

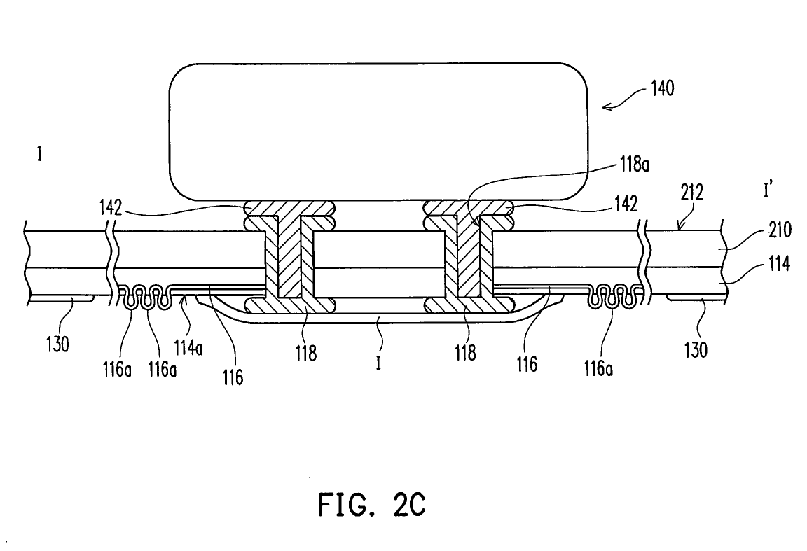

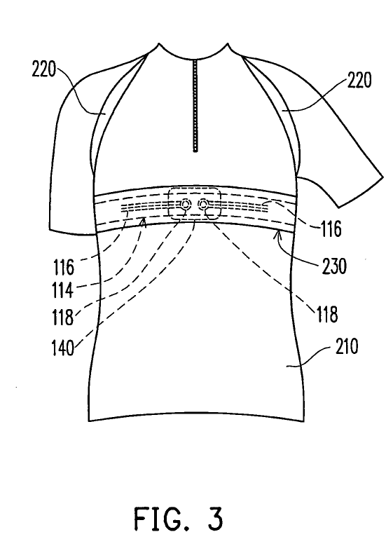

FIG. 2A is a schematic view of a front side of a sports clothes according to another embodiment of the present invention, FIG. 2B is a schematic view of a back side of the sports clothes of FIG. 2A, FIG. 2C is a schematic cross-sectional view of FIG. 2A, taken along a line segment I-I′, and FIG. 3 is a schematic view of a front side of a sports clothes according to still another embodiment of the present invention.

Referring to both FIGS. 2A and 2B, a sports clothes 200 is suitable for being worn on a human body (not shown). The sports clothes 200 includes a main part 210, an elastic reinforcement structure 220, an elastic structure 114, two fabric electrodes 116, and two fasteners 118. The main part 210 may be in a style of a sleeve coat. In addition, it should be noted that the elastic structure 114, the fabric electrodes 116, and the fasteners 118 of this embodiment are the same as the elastic structure 114, the fabric electrodes 116, and the fasteners 118 of FIG. 1C. The elastic reinforcement structure 220 is stitched on a part D of the main part 210 corresponding to a connection part between an arm and a body of the human body, and thus, through the elastic reinforcement structure 220, the main part 210 is not easily slipped or shifted due to being affected by the arms when the human being does sports with the arms.

Referring to FIGS. 2A and 2C, the elastic structure 114 is connected with the main part 210, and considering the connection maimer, the elastic structure 114 of this embodiment is directly machine-sewed or laminated to the main part 210. In an alternative embodiment, the main part 210 has an upper part and a lower part connected with each other by the elastic structure 114.

In addition, in this embodiment, the fasteners 118 penetrate the main part 210 and are exposed out of the surface 212 of the main part 210, in which the surface 212 faces a direction away from the human body. The fasteners 118 are, for example, conductive fasteners, so that an insulating structure I is further disposed to cover the conductive fasteners in this embodiment. In addition, in order to fix the elastic structure 114, an anti-slip structure 130 may be further disposed on the surface 114a of the elastic structure 114 contacting with the human body, so as to prevent the elastic structure 114 from being slipped or shifted while the human being does sports. The anti-slip structure 130 of this embodiment is the same as the anti-slip structure 130 of FIG. 1C.

In addition, in this embodiment, a sensing device 140 is further disposed on the sports clothes 200, which is fixed on the elastic structure 114 through the fasteners 118 and electrically connected with the fabric electrodes 116 through the fasteners 118. The sensing device 140 is, for example, an electrocardiogram sensing device, a respiration sensing device, a heartbeat sensing device, or an electromyography sensing device. In addition, referring to FIG. 3, in order to provide the sports clothes 200 with an elegant appearance, a beautifying structure 230 is stitched or laminated to the main part 210 in this embodiment, so as to cover the sensing device 140. The beautifying structure 230 may be made of the same material as that of the main part 210.

To sum up, the outer layer structure of the present invention completely covers the inner layer structure and the sensing device, so that when observing the sports clothes from the exterior, the sensing device and the inner layer structure cannot be seen at all, and thus the outer layer structure is helpful for beautifying the sports clothes. In addition, through the elastic reinforcement structure in the sports clothes of the present invention, the main part is not easily slipped or shifted due to being affected by the arms when the human being does sports with the arms, thereby enhancing the sensing accuracy of the sensing device disposed on the main part. In the present invention, the beautifying structure is further provided to cover the sensing device, so as to achieve the effect of beautifying the appearance of the sports clothes.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A sports clothes, suitable for being worn on a human body, comprising:
an inner layer structure, comprising:
a main part;
an elastic structure, connected with the main part;
at least two fabric electrodes, directly knitted on the elastic structure, wherein the fabric electrodes are suitable for contacting with skin of the human body; and
at least one fastener, nailed on the elastic structure and electrically connected with the at least two fabric electrodes; and
an outer layer structure, wherein parts of the outer layer structure and that of the inner layer structure corresponding to at least one of shoulder, partial neck portion, and armpit of the human body are connected, and the outer layer structure covers the inner layer structure completely.

2. The sports clothes according to claim 1, further comprising a sensing device, fixed on the elastic structure through the fastener, and electrically connected with the fabric electrodes through the fastener.

3. The sports clothes according to claim 1, wherein the parts of the outer layer structure and that of the inner layer structure corresponding to at least one of the shoulder and the partial neck portion of the human body are directly stitched together.

4. The sports clothes according to claim 1, wherein the part of the outer layer structure and that of the inner layer structure corresponding to the armpit of the human body are connected by point stitching or through an elastic tape.

5. The sports clothes according to claim 1, wherein the elastic structure is directly machine-sewed or laminated to the main part.

6. The sports clothes according to claim 1, wherein the main part of the inner layer structure comprises an upper part and a lower part connected with each other by the elastic structure.

7. The sports clothes according to claim 1, wherein the inner layer structure further comprises an anti-slip structure, covering a surface of the elastic structure.

8. The sports clothes according to claim 7, wherein the anti-slip structure partially covers the surface of the elastic structure or completely covers the surface of the elastic structure.

9. The sports clothes according to claim 1, wherein the fastener is a conductive fastener, and the inner layer structure further comprises an insulating structure, covering the conductive fastener.

10. The sports clothes according to claim 1, wherein a front side of the fastener is a conductive material, and a back side of the fastener is an insulating material.

11. The sports clothes according to claim 1, wherein the inner layer structure is in a style of a sleeveless coat or a vest.

12. The sports clothes according to claim 1, wherein the outer layer structure is in a style of a sleeve coat.

13. A sports clothes, suitable for being worn on a human body, comprising:
a main part;
an elastic reinforcement structure, stitched on a part of the main part corresponding to a connection part between an arm and a body of the human body;
an elastic structure, connected with the main part;
at least two fabric electrodes, directly knitted on the elastic structure, wherein the fabric electrodes are suitable for contacting with skin of the human body; and
at least one fastener, nailed on the elastic structure and electrically connected with the at least two fabric electrodes.

14. The sports clothes according to claim 13, further comprising a sensing device, fixed on the elastic structure through the fastener, and electrically connected with the fabric electrodes through the fastener.

15. The sports clothes according to claim 14, further comprising a beautifying structure, stitched on the main part, so as to cover the sensing device.

16. The sports clothes according to claim 13, wherein the elastic structure is directly machine-sewed or laminated to the main part.

17. The sports clothes according to claim 13, wherein the main part comprises an upper part and a lower part connected with each other by the elastic structure.

18. The sports clothes according to claim 13, further comprising an anti-slip structure, covering a surface of the elastic structure.

19. The sports clothes according to claim 18, wherein the anti-slip structure partially covers the surface of the elastic structure or completely covers the surface of the elastic structure.

20. The sports clothes according to claim 13, wherein the fastener is a conductive fastener, and the sports clothes further comprises an insulating structure, covering the conductive fastener.

21. The sports clothes according to claim 13, wherein a front side of the fastener is a conductive material, and a back side of the fastener is an insulating material.

22. The sports clothes according to claim 13, wherein the main part is in a style of a sleeve coat.
